(19) United States
(12) Patent Application Publication
(10) Pub. No.: US 2026/0224623 A1
(43) Pub. Date: Aug. 6, 2026

(54) Microfluidic System for Transplantation of Mitochondria in Immune Effector Cells

(71) Applicant: Northeastern University, Boston, MA (US)

(72) Inventors: Tania KONRY, Boston, MA (US); Judene THOMAS, Roslindale, MA (US); Augustin DE GANZÓ, Allston, MA (US)

(21) Appl. No.: 19/152,213

(22) PCT Filed: Jan. 29, 2024
(86) PCT No.: PCT/US2024/013408

§ 371 Date: Jul. 30, 2025

Related U.S. Application Data
(60) Provisional application No. 63/441,715, filed on Jan. 27, 2023.

(51) Int. Cl. A61K 35/17; C12N 5/00; C12N 5/0783

(52) U.S. Cl. CPC A61K 35/17; C12N 5/0012; C12N 5/0646; C12N 2502/1164

(57) ABSTRACT

Methods and systems are provided for analyzing the ability of immune cells to kill target cells based on the effectiveness of mitochondria of the immune cells. The systems and methods further can be used for transplanting healthy mitochondria into immune cells to increase their effectiveness against target cells. The methods described herein can be used in treatment of cancer, mitochondrial diseases, and certain metabolic diseases.

### CROSS REFERENCE TO RELATED APPLICATIONS

This application claims the priority of U.S. Provisional Application No. 63/441,715, filed 27 Jan. 2023 and entitled “Microfluidic System for Transplantation of Mitochondria in Immune Effector Cells”, the whole of which is hereby incorporated by reference.

### BACKGROUND

Mitochondria play a crucial role in maintaining cellular balance and providing energy to execute critical cellular functions.¹⁻³ One essential role mitochondria play is in immune cell maintenance by regulating signaling pathways in innate and adaptive immune effector cells to help activate an immune response in addition to other key immune cell functions.^{4,5} Mitochondria can regulate immune cell activation through various metabolic pathways, including oxidative phosphorylation, glycolysis, and production of reactive oxygen species. The use of a particular metabolic pathway by the mitochondria determines the fate of the immune cell and the energy investment required to elicit an immune response via aerobic glycolysis or oxidative phosphorylation.^{6,7} While mitochondrial reactive oxygen species (mtROS) production regulates immune cell activation, excess production of mtROS may lead to mitochondrial dysfunction, hindering the cell death pathway in an otherwise intact mitochondrion, or halting organelle-specific roles altogether.^{8,9} Other factors such as mutations in mitochondrial DNA can also result in dysfunctional mitochondria.^{10,11} Highly functional mitochondria are essential to effective immune cell signaling. Still, mitochondrial dysfunction can be harmful to immune cell activity and has been connected to cancer disease onset and other related immune deficiencies.^{12,13} Metabolic regulation is essential for the activation, growth, and prolonged survival of natural killer (NK) cells.¹⁴

The current degree of variability in immune cell subpopulations poses a challenge to potent and efficacious cancer therapy methods. Addressing this challenge requires broadening the scope of immunotherapy targets to tackle effector cell functional heterogeneity at the single-cell level including the health and prolonged stability of immune cell mitochondria.¹⁷ Understanding how the well-being of mitochondria impact the overall functionality of immune cells is crucial to enhancing cancer therapeutics.

There is a need for methodology to carry out transfer of active mitochondria to recipient cells, such as immune effector cells and other types of cells, that have poor mitochondrial function, so as to improve cell function.

### SUMMARY

The present technology provides methods for replacing or reenergizing cells possessing dysfunctional mitochondria by transplanting functionally active and healthy mitochondria into a dysfunctional or inactive cells, including immune cells. The methods can facilitate a phenotypic switch from an inactive cellular state to a more active state. In the case of immune cells, mitochondrial transplantation can improving an immune system's response to the presence of disease, infection, pathogens, and parasites.

Mitochondrial auto-transplantation involves the extracellular delivery of functional or “sensitive” mitochondria harvested from one cell to another. This technique can be applied, for example, to transfer mitochondria from proficient immune cells to impaired immune cells, thereby enhancing immune cell functionality. In particular, mitochondrial transplantation into NK cells could effectively improve the anti-tumor response of deficient NK subpopulations and increase the effectiveness of NK-based therapies. The present technology utilizes droplet microfluidics for mitochondrial transplantation in single cells and employes omic analysis, including transcriptomics and proteomics using mass spectroscopy and epigenomic.

The present technology can be further summarized with the following list of features.
1. A method for enhancing a function of an immune effector cell, the method comprising the steps of:
   (a) co-encapsulating a first immune effector cell with a first target cell in an aqueous microdroplet and encapsulating a second immune effector cell with a second target cell in another aqueous microdroplet using a microfluidic device;
   (b) incubating the co-encapsulated cells for a period of time in the microfluidic device and observing interaction between the immune effector cells and the target cells, wherein the first immune effector cell is observed to interact more effectively with the first target cell than the second immune effector cell interacts with the second target cell;
   (c) isolating the first immune effector cell and the second immune effector cell; and
   (d) extracting one or more mitochondria from the first immune effector cell and transplanting the extracted mitochondria into the second immune effector cell to obtain an immune transplant cell, whereby the immune transplant cell is more effective at interacting with target cells than the second immune effector cell.
2. The method of feature 1, further comprising:
   (e) incubating the immune transplant cell resulting from step (d) with a third target cell and observing interaction between the immune transplant cell and the third target cell.
3. The method of feature 2, wherein the immune transplant cell is observed to interact more effectively with the third target cell than the second immune effector cell interacted with the second target cell.
4. The method of feature 2 or feature 3, further comprising:
   (f) sequencing mitochondrial DNA from one or more of the first immune effector cell, the second immune effector cell, or the immune transplant cell.
5. The method of any of the preceding features, wherein the observed interactions comprise killing of a target cell.
6. The method of any of the preceding features, wherein step (c) comprises separating the first and second immune effector cells using a droplet sorter that separates microdroplets based on an indicator of effectiveness of interactions between an immune effector cell and a target cell.
7. The method of any of the preceding features, further comprising culturing and expanding the immune transplant cell to form a plurality of progeny of the immune transplant cell in a culture medium.
8. The method of any of the preceding features, wherein any of the first immune effector cell, the second immune effector cell, and the immune transplant cell are selected from the group consisting of T cells, NK cells, CAR T cells, neutrophils, monocytes, and macrophages.
9. The method of any of the preceding features, wherein the target cell is selected from the group consisting of tumor cells, microbial cells, bacteria, and virus-infected cells.
10. The method of any of the preceding features, wherein the first and second immune effector cells are obtained or derived from one individual.
11. The method of any of features 1-9, wherein the first and second immune effector cells are obtained or derived from different individuals.
12. The method of feature 10 or 11, wherein the one or different individuals are humans or non-human mammals.
13. The method of any of the preceding features, wherein a plurality of first and second immune effector cells are used in steps (a)-(d), and wherein a plurality of immune transplant cells are produced in step (d).
14. The method of feature 13, wherein at least 10³, 10⁴, 10⁵, 10⁶, 10⁷, or 10⁸ first and second immune effector cells are used, and wherein at least 10³, 10⁴, 10⁵, 10⁶, 10⁷, or 10⁸ immune transplant cells are produced.
15. A method for transplanting mitochondria into an immune effector cell, the method comprising the steps of:
   (a) providing (i) a microfluidic device configured for co-encapsulating cells in aqueous microdroplets in an oil stream, (ii) a suspension of immune effector cells, and (iii) a suspension of donor mitochondria;
   (b) producing a plurality of aqueous microdroplets from the suspension of immune effector cells and the suspension of mitochondria using the microfluidic device, wherein the plurality of aqueous microdroplets comprises microdroplets comprising both one or more effector cells and one or more donor mitochondria;
   (c) incubating the aqueous microdroplets, whereby donor mitochondria are taken up into immune effector cells to form immune transplant cells; and
   (d) collecting the immune transplant cells.
16. The method of feature 15, further comprising culturing and expanding the collected immune transplant cells to form a plurality of progeny of the immune transplant cells in a culture medium.
17. The method of feature 15 or 16, wherein the immune effector cells are selected from the group consisting of T cells, NK cells, CAR T cells, neutrophils, monocytes, and macrophages.
18. The method of any of features 15-17, wherein the donor mitochondria are autologous or heterologous with respect to the immune effector cells.
19. A method of treating cancer, a mitochondrial disease, or a metabolic disease, in a subject in need thereof, the method comprising performing the method of any of the preceding features, wherein the immune effector cells are obtained or derived from the subject.
20. The method of feature 19, wherein an immune function of the subject is enhanced as a result of said treatment.
21. A system for performing the method of any of the preceding features, the system comprising:
   (a) a microfluidic device configured for encapsulating one or more types of single cells in aqueous microdroplets in an oil stream, incubating the aqueous microdroplets, and observing the incubated aqueous microdroplets with a light microscope;
   (b) a sorting device configured for sorting and recovering individual microdroplets from or within the microfluidic device; and
   (c) reagents for isolating mitochondria.
22. The system of feature 21, further comprising a microdroplet docking array comprising a plurality of rows and columns of docking sites for individual microdroplets and a transparent window configured for light microscopic observation of microdroplets docked in the array.
23. The system of feature 21 or 22, further comprising a plurality of mitochondria for transplantation according to the method of any of features 15-20.

### BRIEF DESCRIPTION OF DRAWINGS

FIG. 1 shows a schematic representation of a general workflow of a mitochondria transplantation process. The process can be used, for example, to improve the toxicity of effector cells for cancer immunotherapy.

FIG. 2 shows a schematic representation of a workflow for immune cell mitochondrial transplantation using a microfluidic device and aqueous microdroplet sorter device.

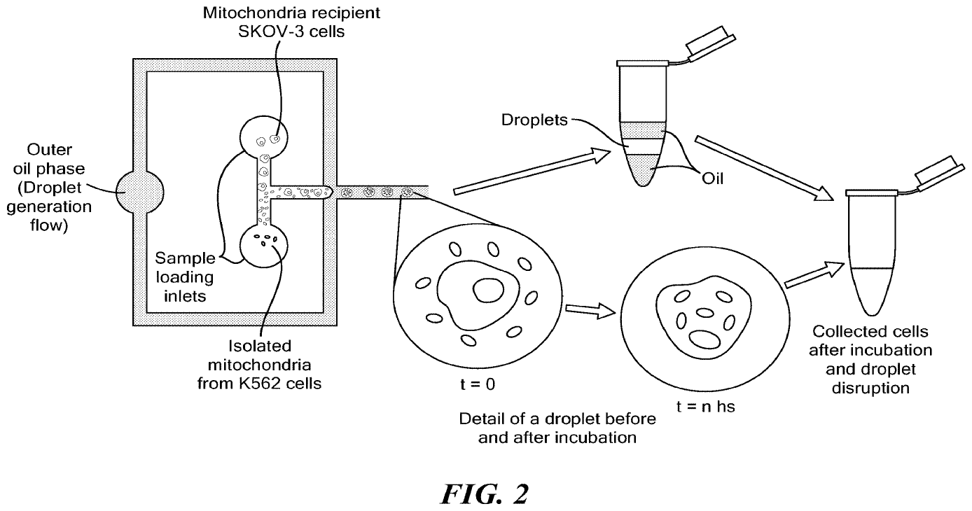

FIG. 3 is a schematic representation of a microfluidic device for mitochondrial transplantation of single cells.

### DETAILED DESCRIPTION

The present technology utilizes microfluidic single-cell technology to co-encapsulate immune cells and target cells in aqueous microdroplets in an oil stream. This system makes it possible to view various parameters of interest, including cytotoxicity, the immune killing of target cells, and drug potency for drug conditional studies.^{18,19} The system allows characterization of dynamic functional responses of single cells and their interactions in real time, for example, when a target tumor cell and an immune effector cell are in the same microenvironment, such as co-localized in an aqueous microdroplet. The methods described herein can be used in particular to analyze and alter effector cell mitochondrial function and makeup using single cell resolution, droplet sorting by a specially adapted droplet sorter device, followed by transcriptomic, proteomic, and epigenomic analysis, including mass spectrometry characterization, to unlock and repair genetic drivers of immune cell regulation and action.

The technology utilizes a microfluidics-based single cell sorting device to co-encapsulate immune cells and target cells in aqueous microdroplets in an oil stream. The device allows the user to evaluate various parameters of interest in the encapsulated cells, including parameters such as cytotoxicity, immune cell killing of target cells, drug potency, and more. In this system, the following steps can be performed to carry out transplantation of mitochondria from one immune cell to another.

The technology can be used to isolate and transplant mitochondria based on any of several specific cell functions that depend on mitochondrial function. For example, mitochondria can be transplanted from drug-sensitive cells or highly functional immune effector cells to drug-resistant cells or functionally impaired immune effector cells, which can increase functionality of an immune response or drug/chemo-sensitivity in cells with newly implanted exogenous mitochondria, and progeny of such cells. This technology can be used in conjunction with an immunotherapy.

A variety of technologies (single cell sorting devices) are available to perform sorting in a microfluidic device of microdroplets containing cells of interest. Known technologies and even commercial kits are available for mitochondrial isolation from cells.

The present technology can access the functional activity of individual effector cells and, where that activity is related to mitochondria of the cells, transfer the mitochondria to other cells with useful properties (e.g., target specificity) but lacking in effectiveness due to defective or poorly functioning mitochondria. The technology also enables genomic analysis of mitochondrial subpopulations that are correlated with the function of specific single cells. The technology allows selection of mitochondria based on the functionality of cell types and sorting of cells with highly functional mitochondria. Selected mitochondria can be isolated and transplanted into cells in order to modulate immune cell activity or functionality based on the introduction of exogenous mitochondria.

The technology can be used with a variety of immune effector cells, either from the same individual donor (autologous) or different donors (heterologous), including natural killer (NK) cells, cells expressing a chimeric antigen receptor (CAR) (e.g., CAR T cells), or other T cells. Cells receiving a mitochondrial transplant can then be propagated and introduced into an individual patient (autologous or heterologous treatment) for enhancing a cell function in the patient, such as to provide more efficient killing of cancer cells or pathogens in the patient.

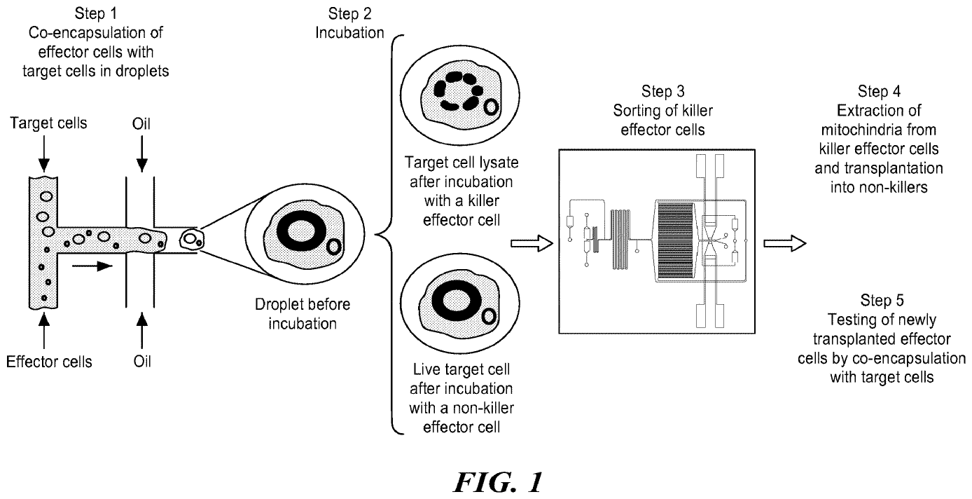

A five step protocol for carrying out cell and mitochondrial selection and transplantation using a single-cell-based microfluidic system is summarized in FIG. 1. The five steps of this protocol are described below.

Step (1). Co-encapsulate an immune cell with a target cell in an aqueous microdroplet using a microfluidic device.

Step (2). Incubate the co-encapsulated cells for a period of time, such as about 6 hours, to allow observation and characterization of function of the immune cell. Light microscopy and optionally fluorescence are used to observe and characterize the actions of the immune cell, including interactions between the immune cell and the target cell.

Step (3). Separate and isolate proven “killers” (immune effector cells observed to have killed target cells, e.g., tumor cells) from “nonkillers” (immune effector cells that have not killed target cells, such as where the target cell remains alive during the incubation) using a sorter module of the microfluidic device.

Step (4). Extract mitochondria from one or more isolated proven killer cells and transplant them into one or more isolated proven nonkiller cells.

Step (5). Incubate the nonkiller cells that have received transplanted mitochondria with one or more target cells and observe whether the nonkiller cells that received transplanted mitochondria have improved killing ability with respect to the target cells.

An optional additional step (Step (6)) includes sequencing the mitochondrial genome of both nonkiller and killer effector cells to assess the differences in the mitochondrial genome associated with cytotoxic activity or other cell functions.

Mitochondrial transplantation involves the delivery of healthy or functional mitochondria into a region of damage.^{20,21} The spontaneous uptake of functional mitochondria by damaged cells occurs through endocytosis and allows for cell “healing” and repair of reversible damage.^{15,21,22} Mitochondrial transfer into damaged or dysfunctional NK cells will improve NK function in populations of NK cells having deficiencies in mitochondrial function. Mitochondrial transplantation at the single-cell level also can provide information about individual NK cells before and after replenishing their mitochondrial population.

In the present technology, autologous or heterologous NK cells or T cells can be obtained from a suitable donor using known methods. After observation in a microfluidic device having a docking array for aqueous microdroplets, the cells can be classified based on function, separating active and/or serial killer cells from less effective or ineffective killer cells using a droplet sorter. Subsequently, mitochondria can be isolated from the active and/or serial killer cells, and then transplanted into ineffective or poorly effective (i.e., “nonkiller”) cells to determine whether the transferal of a mitochondrial subpopulation affects the phenotype of nonkiller cells.

Many different cell types can be characterized and used for mitochondrial transplantation, either as mitochondrial donors or acceptors, using the present technology. These include immune effector cells, such as natural killer cells, CAR T cells, other T cells, neutrophils, monocytes, and macrophages, as well as tumor cells other types of cells. Any type of cell can be characterized and used for mitochondrial transplantation as long as the cell type can be obtained as single cells suitable for introduction into aqueous microdroplets in a microfluidic device. Of particular interest are cells that interact with, and can be co-encapsulated with, other cells in aqueous microdroplets, and cells that have significant energy utilization rates and for which mitochondrial metabolism can be limiting for cellular function.

Mitochondria for transplantation and/or analysis can be obtained from donor cells using a commercial kit. See, e.g., Sun et al., 2022, and kits from Beyotime (Kit C3601) and ThermoFisher (Kit 89801). Briefly, donor cells can be stained using Mito Tracker Green FM (Invitrogen, M7514), the donor cells lysed and homogenized, and the mitochondria isolated by differential centrifugation. For transplantation, the isolated mitochondria, suspended in an aqueous buffer solution, are co-encapsulated with recipient cells in aqueous microdroplets in a microfluidic device. The donor mitochondria are spontaneously taken up by endocytosis into the recipient cells. A variety of assays are available for use to test the effectiveness of mitochondrial transplantation. For example, if the recipient cells are NK cells, whose effectiveness at target cell killing is to be tested after mitochondrial transplantation, then staining of the target cells with propidium iodide (Invitrogen, P3566), a cell death marker, can be used to quantify the level of cell death obtained following co-encapsulation and incubation in aqueous microdroplets in oil in a microfluidic device.

Microfluidic devices capable of co-encapsulating different cell types in microdroplets, or co-encapsulating cells with donor mitochondria in microdroplets, incubating large numbers of cell pairs or groups simultaneously in microdroplet arrays and analyzing their interactions by microscopy, as well as sorting and collection of microdroplets based on cell fluorescence, are known and described, for example, in WO 2018/013726 A1, U.S. Pat. No. 10,718,763 B2, WO 2020/023685 A1, WO 2020/223578 A1, WO 2020/247313 A1, US 2021/0260576 A1, and WO 2023/215608 A1, each of which is hereby incorporated by reference in its entirety.

The present technology provides several novel and advantageous features. It allows determination of the potency of immune cell immunogenicity within a population of immune cells based on mitochondrial makeup of the population. It also permits identification and sorting of highly active immune killer cells from less active immune killer cells. Further, the technology allows the preparation of a population of immune cells, or single cells, having increased immune killing activity at the single-cell level using mitochondrial transplantation. The technology provides methods for identifying mitochondrial subpopulations that differ in their support of immune killer activity, and for determining the underlying features of mitochondrial DNA, including mutations, that underlie functional deficiencies of several types of immune cells. The methods can be used, together with mitochondrial transplantation, to overcome CAR T cell exhaustion in cancer therapy. The methods of the present technology can also be used for profiling of immune cells of individual patients to study the capacity and functionality of the mitochondria of those immune cells. Yet another use of the technology is to perform mitochondrial therapy for patients with immune or metabolic diseases.

### EXAMPLES

### Example 1. Mitochondrial Isolation and Auto-Transplantation Efficacy

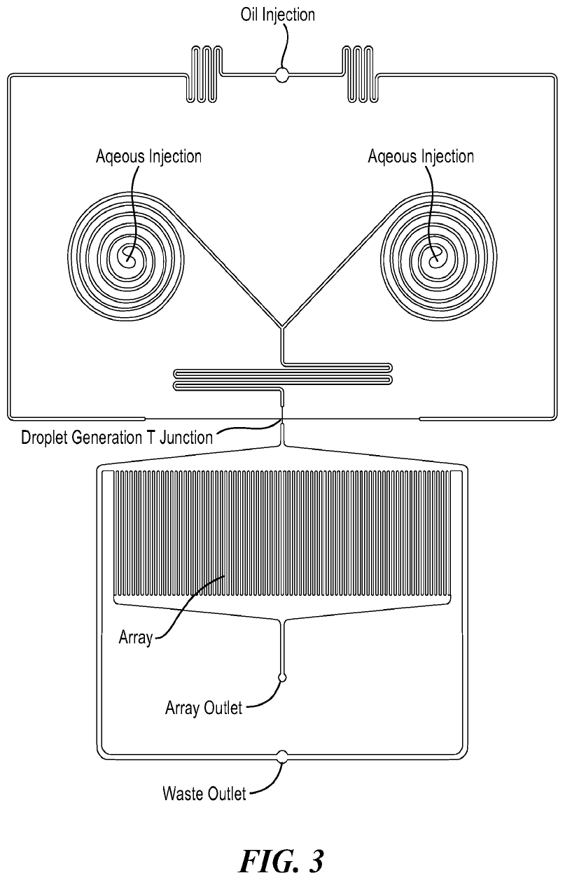

A commercially available mitochondria isolation kit is used for tissue and cell lysates (ThermoFisher, 89801) to harvest mitochondria from donor K562 leukemia cells. To visualize mitochondrial uptake through microscopy, SKOV-3 ovarian adenocarcinoma cells are stained with CellMask Deep Red (Invitrogen, C10046) and mitochondria from donor K562 cells are stained with Mito Tracker Green FM (Invitrogen, M7514). Using the aqueous microdroplet-based microfluidic device depicted in FIG. 3 with an oil injection inlet and two aqueous injection inlets, donor mitochondria and the recipient SKOV-3 cells are introduced through the two aqueous inlets and co-encapsulated into aqueous microdroplets in an oil stream. The cells and mitochondria are co-encapsulated in the droplets for 2 hours at 37° C. to allow for maximal mitochondrial uptake via endocytosis. Following the incubation period, the uptake of donor mitochondria into SKOV-3 cells is confirmed by microscopy. The droplets are then collected and cocultured with NK92 cells, and a plate-based assay is used to assess the sensitivity levels of SKOV-3 transplant cells to NK92 cytotoxicity.

### Example 2. Sensitivity of SKOV-3 to NK92 Killing after Mitochondrial Transplantation into SKOV-3 Cells

The following experiment is designed to determine whether SKOV-3 cells become more susceptible to NK92 cytotoxicity after transplantation with “sensitive” mitochondria isolated from K562 cells. Tumor progression requires a substantial energy supply and tumor cells possess the ability to reprogram mitochondria for their own benefit. During tumor development mitochondria undergo an altered metabolism which may lead to an inactive cell death pathway. Through these alterations, tumor cells are able to evade apoptosis and immune surveillance contributing to their unconstrained proliferation and metastasis.²³

Mitochondrial transplantation is performed using a spiral microfluidic device (FIG. 3) for high throughput mitochondrial uptake through the increase of single-cell co-encapsulation ratio. The donor mitochondria are harvested from K562 cells. This tumor cell line has been proven to be highly susceptible to NK92 cytotoxicity.²⁴ The recipient cell, SKOV-3, a well-established human epithelial ovarian cancer cell line first derived from ovarian adenocarcinoma, has been found to be highly resistant to cytotoxic drugs.²⁵ NK92 cells are interleukin-dependent NK cells and have been rigorously used for immune cell studies for inducing cell mediated.

A plate-based LDH Coculture Assay is to test susceptibility of target tumor cells to NK92 cytotoxicity. A statistically significant difference in sensitivity to NK92 cytotoxicity between the target tumor cells is evidence that mitochondrial transfer can induce a distinct shift in tumor phenotypic response upon re-exposure to immune cell cytotoxicity. After a 2-hour coculture of SKOV-3 with NK92 cells in microdroplets, SKOV-3 cell viability is measured. Cells are recovered from droplets and stained with propidium iodide (Invitrogen, P3566), a cell death marker, to quantify the level of cell death following mitochondrial transplantation.

### Example 3. Detection of a Mitochondrial Subpopulation in Tumors that Drive Tumor Proliferation and Metastasis

Tumor cells have been shown to hijack mitochondria for their own energy needs, using mitochondrial oxidative phosphorylation to promote tumor proliferation and expansion to other cellular environments. Tumor cells can also evade mitochondrially-engineered cell death (apoptosis) through various processes.⁸ Here, tumor cells are transplanted with “sensitive” mitochondria (mitochondria with an intact cell death pathway). The sensitivity of the tumor cells to NK-induced killing and drug-induced apoptosis is measured following transplantation of mitochondria into the tumor cells.

### Example 4. Asessment of the Role of Mitochondrial Metabolism in NK Cell Aging, Functionality, and Cytotoxicity Towards Target Cells

Using a microfluidic device with an integrated sorter module, serial killing NK cells are separated from less active NK cells based on an increased florescence signal that is produced when a target tumor cell is killed. Less active NK cells are used for auto-transplantation with mitochondria from highly active NK cells. Individual NK cells are analyzed for their killing ability before and after mitochondrial transplantation to determine if a particular cell's response is based on function of a mitochondrial subpopulation. Overall, droplet microfluidics can help us elucidate the role of NK and T cell metabolism in cancer development through single-cell isolation of immune cells that exogenous functional mitochondria have modified. The profiling of immune cells using this approach in patients can increase the efficacy of mitochondrial therapy as a high throughput method of treatment for patients with cancer, as well as mitochondrial and other metabolic diseases.

Aqueous microdroplets are generated with co-encapsulated NK92 immune cells and K562 tumor target cells. The droplets are incubated for 6 hours at 37 C to facilitate killing events (single cell killing, serial killing, and synaptic contact). Target tumor cell death is determined based on a standard apoptosis cell dye. Tumor cells are stained with CellEvent Caspase 3/7 apoptosis dye (Invitrogen, C10423) to detect killing events. Following incubation, a droplet sorter device is used to sort droplets into active (fast, serial killing) NK cells and inactive NK cells based on their killing ability for K562 tumor cells as target cells. When an active or serial NK cell kills a k562 that has been stained with an apoptosis dye, a fluorescent signal is produced which activates the sorter to direct the droplets containing active NK cells to a collection channel. The non-sorted droplets which contain inactive or non-killing NKs are collected in a separate “waste” channel. Both sets of droplets are collected for downstream processing. The isolated fast or serial killing NKs are recovered from droplets and expanded in culture. An LDH coculture assay is then carried out on sorted NK killer cells to establish whether expanded killer NK cells possess higher cytotoxicity compared to a heterogenous population of NK92 cells. the LDH coculture assay is repeated on expanded killer cells (after at least 2 weeks) to establish whether the expanded cells have approximately the same cytotoxicity as the originally sorted killer cells. Extracted mitochondria from the sorted and the later expanded killer NK cells are transplanted into inactive NK cells. These new NK transplant cells are then cocultured with target tumor cells in a microdroplet array device to determine if the killing ability of transplant cells has improved.

### Example 5. Determination of Functional Interactomics of Potent Killer T Cells

Transcriptomics analysis using RNA sequencing is performed to determine what transcription factors are upregulated along specific pathways during an immune response by individual highly effective killer cells. Proteomics is also performed using mass spectrometry of the same cells to determine the most active proteins at the point of immune activation. Epigenetic analysis also is performed to determine changes in gene activity upon immune cell activation and killing. Biomarkers for the most effective killer cells are identified.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

### REFERENCES

(1) Klein, K.; He, K.; Younes, A. I.; Barsoumian, H. B.; Chen, D.; Ozgen, T.; Mosaffa, S.; Patel, R. R.; Gu, M.; Novaes, J. et al. Role of Mitochondria in Cancer Immune Evasion and Potential Therapeutic Approaches. Front Immunol 2020, 11, 573326.
(2) Brand, M. D.; Orr, A. L.; Perevoshchikova, I. V.; Quinlan, C. L. The role of mitochondrial function and cellular bioenergetics in ageing and disease. Br J Dermatol 2013, 169 Suppl 2 (0 2), 1.
(3) Chen, W.; Zhao, H.; Li, Y. Mitochondrial dynamics in health and disease: mechanisms and potential targets. Signal Transduct Target Ther 2023, 8 (1), 333.
(4) Weinberg, S. E.; Sena, L. A.; Chandel, N. S. Mitochondria in the regulation of innate and adaptive immunity. Immunity 2015, 42 (3), 406.
(5) Song, Y.; Zhou, Y.; Zhou, X. The role of mitophagy in innate immune responses triggered by mitochondrial stress. Cell Commun Signal 2020, 18 (1), 186.
(6) Bahat, A., MacVicar, T., & Langer, T. Metabolism and Innate Immunity Meet at the Mitochondria. Frontiers in Cell and Developmental Biology 2021, 9, 720490.
(7) Chen, Y.; Zhou, Z.; Min, W. Mitochondria, Oxidative Stress and Innate Immunity. Front Physiol 2018, 9, 1487.
(8) Warburg, O. On the origin of cancer cells. Science 1956, 123 (3191), 309.
(9) Li, X. Y.; Fang, P.; Mai, J. T.; Choi, E. T.; Wang, H.; Yang, X. F. Targeting mitochondrial reactive oxygen species as novel therapy for inflammatory diseases and cancers. Journal of Hematology & Oncology 2013, 6.
(10) Taylor, R. W.; Turnbull, D. M. Mitochondrial DNA mutations in human disease. Nat Rev Genet 2005, 6 (5), 389.
(11) Smith, A. L. M.; Whitehall, J. C.; Greaves, L. C. Mitochondrial DNA mutations in ageing and cancer. Mol Oncol 2022, 16 (18), 3276.
(12) Porporato, P. E.; Filigheddu, N.; Pedro, J. M. B.; Kroemer, G.; Galluzzi, L. Mitochondrial metabolism and cancer. Cell Res 2018, 28 (3), 265.
(13) Wang, S. F.; Tseng, L. M.; Lee, H. C. Role of mitochondrial alterations in human cancer progression and cancer immunity. J Biomed Sci 2023, 30 (1), 61.
(14) Terren, I.; Orrantia, A.; Vitalle, J.; Astarloa-Pando, G.; Zenarruzabeitia, O.; Borrego, F. Modulating NK cell metabolism for cancer immunotherapy. Semin Hemato/2020, 57 (4), 213.
(15) McCully, J. D.; Levitsky, S.; Del Nido, P. J.; Cowan, D. B. Mitochondrial transplantation for therapeutic use. Clin Transl Med 2016, 5 (1), 16.
(16) Desdin-Mico, G.; Soto-Heredero, G.; Mittelbrunn, M. Mitochondrial activity in T cells. Mitochondrion 2018, 41, 51.
(17) Zhang, Y. Y.; Zhang, Z. M. The history and advances in cancer immunotherapy: understanding the characteristics of tumor-infiltrating immune cells and their therapeutic implications. Cell Mol Immunol 2020, 17 (8), 807.
(18) S Sarkar, P. S., D Stroopinsky, K Palmer, N Cohen, J Rosenblatt, D Avigan, T Konry. Dynamic analysis of immune and cancer cell interactions at single cell level in microfluidic droplets. Biomicrofluidics 2016, 10, 054115.
(19) Agnihotri, S. N.; Ugolini, G. S.; Sullivan, M. R.; Yang, Y.; De Ganzo, A.; Lim, J. W.; Konry, T. Droplet microfluidics for functional temporal analysis and cell recovery on demand using microvalves: application in immunotherapies for cancer. Lab Chip 2022, 22 (17), 3258.
(20) Liu, Z. H.; Sun, Y.; Qi, Z. T.; Cao, L.; Ding, S. Z. Mitochondrial transfer/transplantation: an emerging therapeutic approach for multiple diseases. Cell Biosci 2022, 12 (1).
(21) Mccully, J. D.; del Nido, P. J.; Emani, S. M. Mitochondrial transplantation: the advance to therapeutic application and molecular modulation. Front Cardiovasc Med 2023, 10.
(22) Liu, D. L.; Gao, Y. S.; Liu, J.; Huang, Y. G.; Yin, J. H.; Feng, Y. Y.; Shi, L. J.; Meloni, B. P.; Zhang, C. Q.; Zheng, M. H. et al. Intercellular mitochondrial transfer as a means of tissue revitalization. Signal Transduct Tar 2021, 6 (1).
(23) Ghosh P, V. C., Dey S, Zhang L. Mitochondria Targeting as an Effective Strategy for Cancer Therapy. Int J Mol Sci May 9, 2020, 21(9):3363.
(24) Song, X.; Xu, C. F.; Wu, X. L.; Zhao, X.; Fan, J. P.; Meng, S. F. The potential markers of NK-92 associated to cytotoxicity against K562 cells. Biologicals 2020, 68, 46.
(25) Gonzalez, V. D.; Huang, Y. W.; Delgado-Gonzalez, A.; Chen, S. Y.; Donoso, K.; Sachs, K.; Gentles, A. J.; Allard, G. M.; Kolahi, K. S.; Howitt, B. E. et al. High-grade serous ovarian tumor cells modulate NK cell function to create an immune-tolerant microenvironment. Cell Reports 2021, 36 (9).
(26) Klingemann, H.; Boissel, L.; Toneguzzo, F. Natural Killer Cells for immunotherapy-Advantages of the NK-92 Cell Line over Blood NK Cells. Frontiers in Immunology 2016, 7.
(27) Sun J, L. H., Fan L, Yiu TL, Shakoor A, Li G, Lee WYW, Sun D. High-efficiency quantitative control of mitochondrial transfer based on droplet microfluidics and its application on muscle regeneration. Sci Adv 2022 Aug. 19, 8(33):eabp9245.

What is claimed is:

1. A method for enhancing a function of an immune effector cell, the method comprising the steps of:
(a) co-encapsulating a first immune effector cell with a first target cell in an aqueous microdroplet and encapsulating a second immune effector cell with a second target cell in another aqueous microdroplet using a microfluidic device;
(b) incubating the co-encapsulated cells for a period of time in the microfluidic device and observing interaction between the immune effector cells and the target cells, wherein the first immune effector cell is observed to interact more effectively with the first target cell than the second immune effector cell interacts with the second target cell;
(c) isolating the first immune effector cell and the second immune effector cell; and
(d) extracting one or more mitochondria from the first immune effector cell and transplanting the extracted mitochondria into the second immune effector cell to obtain an immune transplant cell, whereby the immune transplant cell is more effective at interacting with target cells than the second immune effector cell.

2. The method of claim 1, further comprising:
(e) incubating the immune transplant cell resulting from step (d) with a third target cell and observing interaction between the immune transplant cell and the third target cell.

3. The method of claim 2, wherein the immune transplant cell is observed to interact more effectively with the third target cell than the second immune effector cell interacted with the second target cell.

4. The method of claim 2, further comprising:
(f) sequencing mitochondrial DNA from one or more of the first immune effector cell, the second immune effector cell, or the immune transplant cell.

5. The method of claim 1, wherein the observed interactions comprise killing of a target cell.

6. The method of claim 1, wherein step (c) comprises separating the first and second immune effector cells using a droplet sorter that separates microdroplets based on an indicator of effectiveness of interactions between an immune effector cell and a target cell.

7. The method of claim 1, further comprising culturing and expanding the immune transplant cell to form a plurality of progeny of the immune transplant cell in a culture medium.

8. The method of claim 1, wherein any of the first immune effector cell, the second immune effector cell, and the immune transplant cell are selected from the group consisting of T cells, NK cells, CAR T cells, neutrophils, monocytes, and macrophages.

9. The method of claim 1, wherein the target cell is selected from the group consisting of tumor cells, microbial cells, bacteria, and virus-infected cells.

10. The method of claim 1, wherein the first and second immune effector cells are obtained or derived from one individual.

11. The method of claim 1, wherein the first and second immune effector cells are obtained or derived from different individuals.

12. The method of claim 11, wherein the different individuals are humans or non-human mammals.

13. The method of claim 1, wherein a plurality of first and second immune effector cells are used in steps (a)-(d), and wherein a plurality of immune transplant cells are produced in step (d).

14. The method of claim 13, wherein at least 10³, 10⁴, 10⁵, 10⁶, 10⁷, or 10⁸ first and second immune effector cells are used, and wherein at least 10³, 10⁴, 10⁵, 10⁶, 10⁷, or 10⁸ immune transplant cells are produced.

15. A method for transplanting mitochondria into an immune effector cell, the method comprising the steps of:
(a) providing (i) a microfluidic device configured for co-encapsulating cells in aqueous microdroplets in an oil stream, (ii) a suspension of immune effector cells, and (iii) a suspension of donor mitochondria;
(b) producing a plurality of aqueous microdroplets from the suspension of immune effector cells and the suspension of mitochondria using the microfluidic device, wherein the plurality of aqueous microdroplets comprises microdroplets comprising both one or more effector cells and one or more donor mitochondria;
(c) incubating the aqueous microdroplets, whereby donor mitochondria are taken up into immune effector cells to form immune transplant cells; and
(d) collecting the immune transplant cells.

16. The method of claim 15, further comprising culturing and expanding the collected immune transplant cells to form a plurality of progeny of the immune transplant cells in a culture medium.

17. The method of claim 15, wherein the immune effector cells are selected from the group consisting of T cells, NK cells, CAR T cells, neutrophils, monocytes, and macrophages.

18. The method of claim 15, wherein the donor mitochondria are autologous or heterologous with respect to the immune effector cells.

19. A method of treating cancer, a mitochondrial disease, or a metabolic disease, in a subject in need thereof, the method comprising performing the method of claim 1, wherein the immune effector cells are obtained or derived from the subject.

20. The method of claim 19, wherein an immune function of the subject is enhanced as a result of said treatment.

21. A system for performing the method of claim 1, the system comprising:
(a) a microfluidic device configured for encapsulating one or more types of single cells in aqueous microdroplets in an oil stream, incubating the aqueous microdroplets, and observing the incubated aqueous microdroplets with a light microscope;
(b) a sorting device configured for sorting and recovering individual microdroplets from or within the microfluidic device; and
(c) reagents for isolating mitochondria.

22. The system of claim 21, further comprising a microdroplet docking array comprising a plurality of rows and columns of docking sites for individual microdroplets and a transparent window configured for light microscopic observation of microdroplets docked in the array.

23. The system of claim 21, further comprising a plurality of mitochondria for transplantation.
